(19) United States
(12) Patent Application Publication
(10) Pub. No.: US 2022/0072383 A1
(43) Pub. Date: Mar. 10, 2022

(54) INTERACTIVE EXERCISE APPARATUS

(71) Applicant: Johnson Health Tech. Co., Ltd., Taichung City (TW)

(72) Inventors: Hung-Mao Liao, Taichung City (TW); Joe Chen, Taichung City (TW); Cheng-Kun Lee, Taichung City (TW); Hsin-Huang Chiang, Taichung City (TW); Yu-Chieh Lee, Taichung City (TW)

(73) Assignee: Johnson Health Tech. Co., Ltd.

(21) Appl. No.: 17/468,690

(22) Filed: Sep. 8, 2021

(30) Foreign Application Priority Data
Sep. 9, 2020 (CN) 202010940057.5

(51) Int. Cl. A63B 24/00; A63B 71/06

(52) U.S. Cl. CPC A63B 24/0084; A63B 71/0622; A63B 2225/12; A63B 2024/0068; A63B 2071/0638; A63B 2220/807

(57) ABSTRACT

A dynamic ranking system for showing ranking information when a user participates in an exercise class includes an exercise database, a mirror display device, a motion capturing unit and a comparing unit. The exercise database is configured to store exercise scores of participants who have participated in the exercise class. The mirror display device has a mirror, a camera device and a display device on a backside of the mirror for showing video content which includes a guiding image for guiding the user to perform a workout when the user participates in the exercise class. The motion capturing unit is configured to capture movement of the user. The comparing unit is configured to determine a matching degree between the movement of the user and movement of the guiding image to obtain an exercise score of the user, and to identify a grade of the user according to the exercise score.

### BACKGROUND

### 1. Field of the Invention

The present invention relates to an interactive exercise apparatus. More particularly, the present invention relates to a dynamic leaderboard and dynamic ranking system for online exercises.

### 2. Description of the Related Art

Indoor exercise is becoming more and more popular in these days. More people are becoming aware of the need to exercise in order to maintain or improve their health and fitness. Since a person's schedule, weather, or other factors may prohibit the person from exercising outdoors, it is more convenient for the person to exercise indoors. Accordingly, indoor exercise apparatuses such as treadmills, stair exerciser apparatuses, steppers, exercise bikes provide such advantages for the person to use.

There is a smart mirror product on the market, which can display demonstration images on the mirror, as a fitness instructor provides real-time instructions. When the user stands in front of the fitness mirror, the user can see their mirror image and follow the demonstration images to perform workout, such that the user can correct their motion or posture immediately. Specifically, the smart mirror is provided for allowing the user to perform online exercise classes.

However, when performing the online exercise classes at home, there are generally no other exercisers around. It may lack of competitiveness, and easily lack the motivation to exercise continuously.

The present invention has arisen to mitigate and/or obviate the disadvantages of the conventional method. Further benefits and advantages of the present invention will become apparent after a careful reading of the detailed description with appropriate reference to the accompanying drawings.

### SUMMARY

The present invention is directed to a dynamic ranking system for online exercise classes. Users can participate in the online exercise classes through a plurality of interactive exercise apparatuses. The system is able to provide leaderboard information and ranking information for the users, so that the users are competitive and motivated to participate in exercise classes actively and continuously.

According to one aspect of the present invention, a dynamic ranking system for showing ranking information when a user participates in an exercise class includes an exercise database, a mirror display device, a motion capturing unit and a comparing unit. The exercise database is configured to store exercise scores of a plurality of participants who have participated in the exercise class. The mirror display device has a mirror, a camera device mounted in the mirror display device and a display device disposed on a backside of the mirror for showing video content and being visible through the mirror. The video content includes a guiding image for guiding the user to perform a workout when the user participates in the exercise class. The motion capturing unit is configured to capture movement of the user through the camera device. The comparing unit is configured to determine a matching degree between the movement of the user and movement of the guiding image to obtain an exercise score of the user, and to identify a grade of the user according to the exercise score.

Preferably, the comparing unit is configured to rank the exercise scores of all participants in the exercise class into at least three levels, and display the level to which the current exercise score of the user belongs on the mirror display device. Specifically, the exercise scores of all participants may be divided into four levels including: a first level where the exercise score is above 75th percentile of all participants, a second level where the exercise score is between 50th percentile and 75th percentile of all participants, a third level where the exercise score is between 25th percentile and 50th percentile of all participants, and a fourth level where the exercise score is below 25th percentile of all participants.

Preferably, the guiding image includes an instructor image demonstrating exercise movements of the exercise class to allow the user to follow and/or a guide track for guiding the user to perform exercise movements of the exercise class.

Preferably, the exercise class has a plurality of sections, and the exercise score of the user is based on accumulation of exercise scores of the sections in the exercise class the user has finished.

Preferably, the mirror display device is configured to display the matching degree of the user in a dynamic action scoring area, a ranking of the user in a dynamic ranking area, and the grade of the user in a dynamic grade area. The ranking of the user is a ranking of their exercise score compared with exercise scores of other participants in the exercise class.

Further benefits and advantages of the present invention will become apparent after a careful reading of the detailed description with appropriate reference to the accompanying drawings.

### BRIEF DESCRIPTION OF THE DRAWINGS

FIG. 1 is a schematic diagram illustrating a system for online exercise classes;

FIG. 2 illustrates two smart mirrors can be interconnected with each other;

FIG. 3 illustrates an smart mirror provided for guiding a user to perform an online exercise class;

FIGS. 4-5 show a display interface of the smart mirror shown in FIG. 3;

FIG. 6 to FIG. 8 illustrate displacement in a dynamic ranking area when rankings of exercisers is raised and fallen;

FIG. 9A illustrates calculation method of the exercise score;

FIG. 9B shows an example of calculation of the exercise score;

FIG. 10 shows ranking rules for a dynamic ranking system;

FIG. 11 shows the system sends a notification to the user on their portable device;

FIG. 12 shows a workout result on an interface of the smart mirror;

FIG. 13 shows ranking information of the user in an online exercise class they have participated; and

FIG. 14 shows exercise records of the user within a specific time range.

### DETAIL DESCRIPTION

The present invention provides a dynamic leaderboard and a dynamic ranking system for online exercises, allowing multiple people to participate in an online exercise class (a physical exercise class, a fitness class, etc.) through a plurality of interactive exercise apparatuses (or smart mirrors) at the same time or at different times, and providing leaderboard information and ranking information according to exercise scores (or workout scores) of the participants, which will be displayed on a display interface.

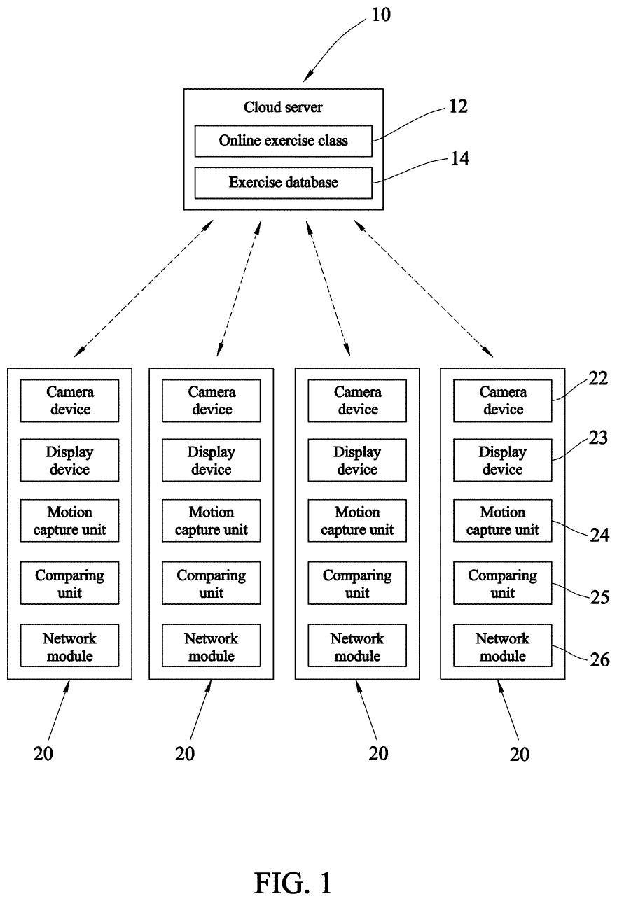

Referring to FIG. 1, in a preferred embodiment of the present invention, a cloud server 10 is configured to provide a plurality of online exercise classes 12 and an exercise database 14. The exercise classes 12 can be accessed by one or more participants who want to participate in one of the exercise classes 12. Each exercise class 12 can be opened at a specific timer period, for example: start the exercise class 12 at 7 PM every Monday, Wednesday and Friday; or, each exercise class 12 can be reserved by one or more participants through the cloud server 10 (or a network server), such that the exercise class 12 can be started at a scheduled time; or users can participate in various exercise classes 12 at any time.

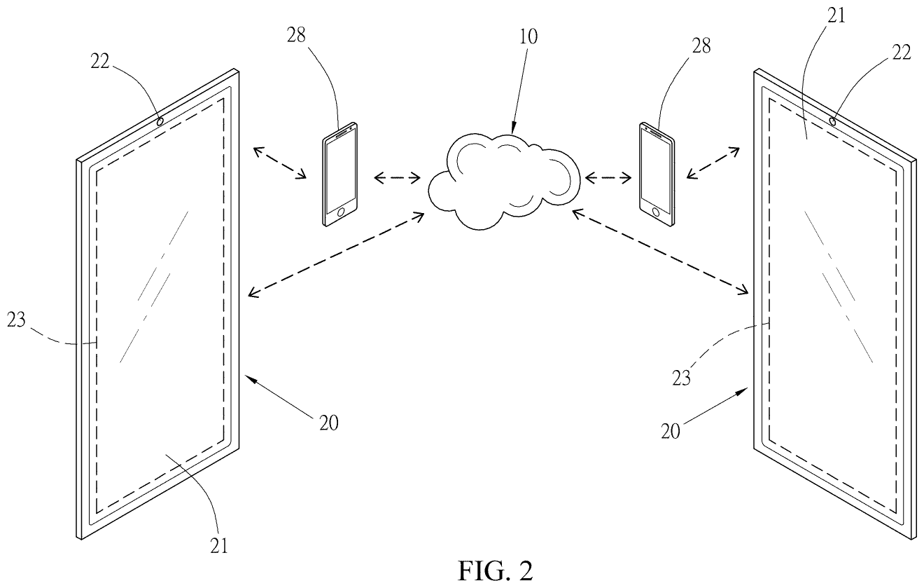

Referring to FIG. 2, each user (or participant) can connect to the cloud server 10 through a smart mirror 20 and a portable device 28 (e.g. a smart phone, a smart watch, a tablet, a computer), to perform an exercise class 12 that they choose to participate in. In the preferred embodiment of the present invention, the portable device 28 is a smart phone. Both the smart mirror 20 and the portable device 28 can connect to the cloud server 10, and the smart mirror 20 and the portable device 28 can also be connected with each other.

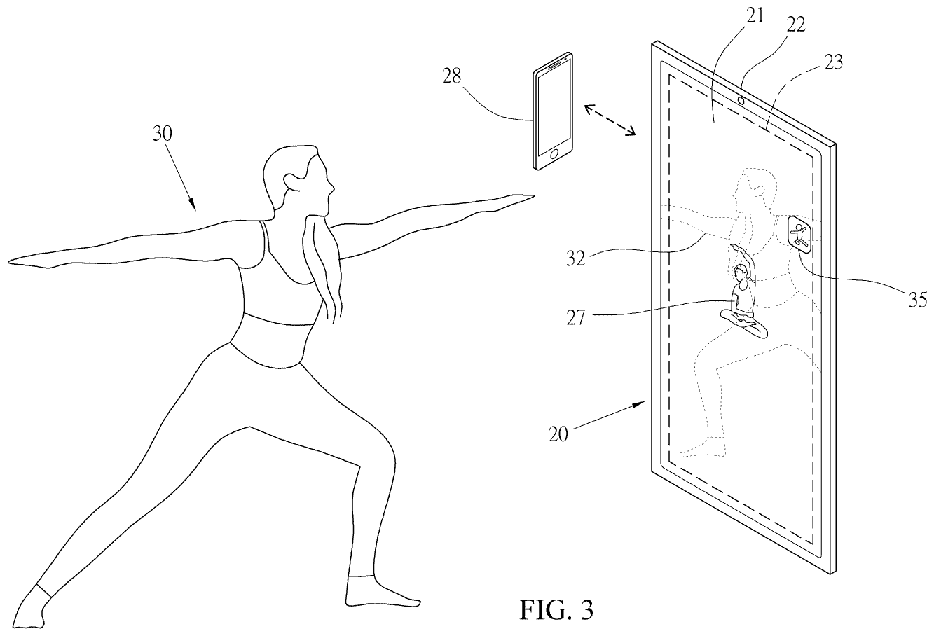

Referring to FIG. 1 and FIG. 3, the smart mirror 20 has a frame, a mirror 21, a camera device 22, a display device 23, a motion capture unit 24, a comparing unit 25, a network module 26, a control unit (not shown), and a voice device (not shown). The mirror 21 is mounted on the frame and the display device 23 is disposed on the backside of the mirror 22. The mirror 21 is configured to reflect an image of a user 30 opposite the mirror 21, so that the user 30 is able to see their mirror image 32 in the mirror 21. The display device 23 is configured to display video content including an instructor image 27 demonstrating movements of an exercise class, and information related to exercise classes. The mirror 21 is a half mirror or a transparent mirror, such that the video content displayed on the display device 23 is visible through the mirror 21.

The control unit regards as a computing and control core, which can be electrically connected to the camera device 22, the display device 23, the motion capture unit 24, the comparing unit 25, the network module 26 and the voice device. The control unit is operable to control operation of the smart mirror 20. When performing an online exercise, the user 30 is able to see the instructor image 27 and their mirror image 32 on the mirror 21 of the smart mirror 20 at the same time.

The network module 26 is provided for allowing the smart mirror 20 to be connected to an external network, including local area network (LAN) and wide area network (WAN), to download data from a local or remote database, including exercise programs, and store in a storage device. Conversely, data can also be uploaded to the aforementioned database via the network, such as uploading fitness data of users. The smart mirror 20 can also be in communication with other smart mirrors or other portable devices 28 via the network module 26, forming a wired or wireless bidirectional communication connection, so that the smart mirror 20 can transmit specific data to the portable device 28 and/or receive specific data from the portable device 28. The smart mirror 20 can allow the user to invite their friends to take exercise classes online, and the mirror 21 of the smart mirror 20 can display the real-time image of at least one friend (namely friend image 35) thereon so as to increase interactivity when participating in an online exercise class. The mirror 21 of the smart mirror 20 may be a touch screen, so that the user is able to input commands by touch control.

Referring to FIG. 2, two smart mirrors 20 can be interconnected with each other. For example, the user's friend may also have a smart mirror which can be wirelessly connected with a portable device (or a smart phone). The user is able to connect with the friend's smart mirror through their smart mirror 20, or the user can connect with the friend's portable device or smart mirror through their portable device 28. The portable devices 28 each have an application program (namely a mobile application, also referred to as a mobile app or simply an app) for allowing the user to control operation of the respective smart mirrors 20, so that the user is able to invite their friends to join a specific exercise class via the application program of the portable device 28.

In the preferred embodiment of the present invention, the exercise database 14 can be built in the aforementioned cloud server 10 to collect and record exercise scores of all participants in each online exercise class, including exercise scores during the exercise class or after the exercise class. The participants are all users who have participated in the respective exercise class. For example, when a user participates in an online exercise class, the data recorded in the exercise database 14 includes exercise data of the user and other participants during the exercise class or after the exercise class. The exercise data includes: exercise score, elapsed time, exercise history, burned calories, heart rate, and so on.

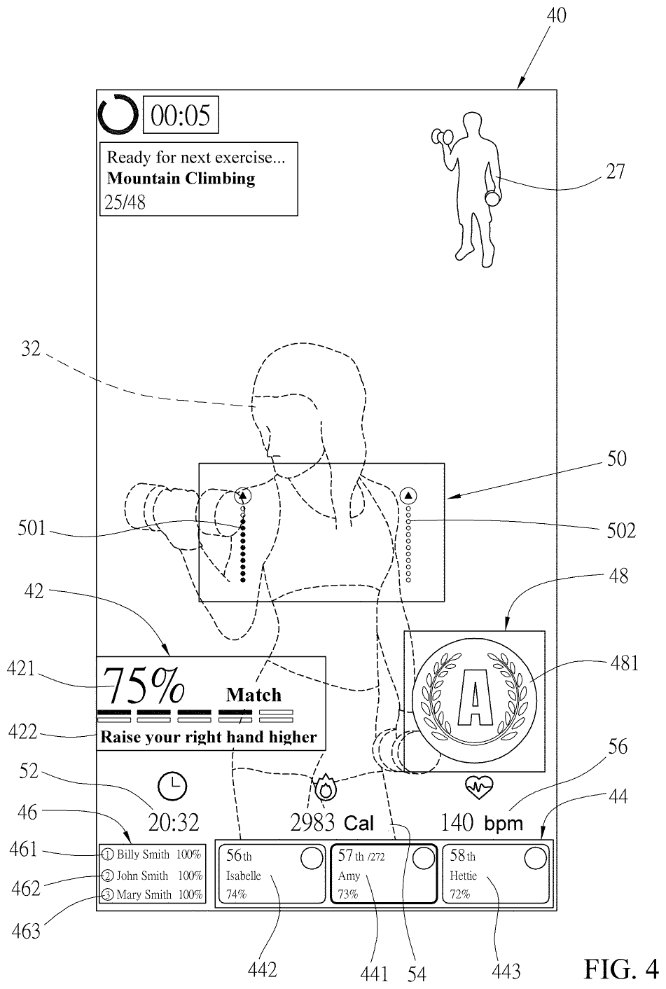

FIG. 4 and FIG. 5 show a display interface 40 on the display device 23 of the dynamic ranking system of the present invention. The display interface 40 is arranged with: an instructor image 27 of an exercise class, a dynamic action scoring area 42, a dynamic ranking area 44, a dynamic leaderboard area 46, a dynamic grade area 48, etc., showing information related to exercise ranking and exercise grade. The display interface 40 also includes a motion guide area 50 with at least one guide track for guiding the user to perform exercise movements of the exercise class. In general, the instructor image 27, the action scoring area 42, the ranking area 44, the leaderboard area 46 and the grade area 48 are displayed on the display interface 40 during the exercise class or after the exercise class, and the user's mirror image reflected by the mirror 21 of the smart mirror 20 are also shown in the mirror 21 at the same time.

When the online exercise class 12 starts, the user follows the movement demonstrated by the instructor image 27 to perform the exercise and the motion capture unit 24 of the smart mirror 20 can dynamically capture movement of the user while exercising through the camera device 22. The comparing unit 25 is configured to compare the movement of the user and the movement of the instructor image 27 to determine a matching degree therebetween. By determining the matching degree between the movement of the user and the movement of the instructor image 27, a real-time exercise score 421 (or matching degree) is generated in the action scoring area 42, as shown in FIG. 4 and FIG. 5. In the preferred embodiment, the exercise score 421 is expressed as a percentage. As shown in FIG. 4, the exercise score 421 of the user is 75%. The motion capture unit 24 and the comparing unit 25 capture and compare the motion of the user with instructor image 27 in real time, such that the exercise score 421 is a dynamic, real-time score. The exercise score 421 will vary with the accuracy and matching degree of the user's movement during the exercise class. The exercise score 421 can be computed by the control unit of the smart mirror 20; or the data obtained by the motion capture unit 24 and the comparing unit 25 can be uploaded to the cloud server 10, and the exercise score 421 can be computed by the cloud server 10 and transmit it to the smart mirror 20 thereafter. Furthermore, the comparing unit can also be installed in the cloud server 10, not limited to being installed in the smart mirror 20.

When the online exercise class 12 starts, the motion guide area 50 is programed to show the guide track for guiding movement of the user so as to increase accuracy of the movement. For example, when performing a fitness class of alternating dumbbell curls as shown in FIG. 4, the user alternately curls dumbbells with both hands, and the motion guide area 50 will show two guide tracks 501, 502 corresponding to predetermined body parts of the user, namely two hands. The corresponding guide track can instruct the user's hand to perform curling motion, and showing feedback information about movement of the user, namely showing whether the degree of curling is in line with the predetermined track by a metering bar or other metering means. Each guide track has an increment or decrement value. When the metering value increases, it means that the user's motion is more in line with the predetermined track. Each guide track can be a light strip, or it can be a light signal with a plurality of small circles arranged in a straight line as shown in FIG. 4. The increase or decrease of the light signal represents the matching degree of the user's movement with respect to the instructor's movement. The metering value is obtained by the operation of the motion capture unit 24 and the comparing unit 25.

Referring to FIG. 4, the guide track 501 indicates the user to raise right hand to increase accuracy of the movement. In addition to the guide tracks 501, 502, the present invention can also use guiding text 422 on the mirror 21 of the smart mirror 20 to indicate motion of the user. For example, using the guiding text 422 of “raise your right hand higher” in the action scoring area 42 so as to increase the matching degree of the user's movement with respect to the instructor image 27, and increase the exercise score 421.

When the online exercise class is for lower limb exercises, such as squats, the motion guide area 50 shows a guide track for guiding the user to do squats, and showing whether the degree of squat is in line with a predetermined movement by a metering bar. The motion guide area 50 may be a floating area to facilitate movement of the motion guide area 50 on the mirror 21 to the position of the corresponding body part of the user. For example, when the user performs a squat exercise, the motion guide area 50 moves to the user's leg portion with corresponding guide tracks; when the user performs a dumbbell exercise, the motion guide area 50 moves to the user's hand portion with corresponding guide tracks 501, 502.

The dynamic ranking area 44 is a real-time ranking area to display the user's exercise ranking information. Preferably, the dynamic ranking area 44 shows the rankings of at least three exercisers. In the preferred embodiment of the present invention, three small blocks (ranking blocks) are used to display ranking information of three exercisers, including: the user himself, at least one exerciser who is ranked in front of the user, at least one exerciser who is ranked behind the user, and the exercise ranking and the exercise score of each exerciser. In general, the user is located in the center of the dynamic ranking area 44 between the two exercisers. As shown in FIG. 4, the user (Amy) 441 is ranked 57th out of 272 participants. The dynamic ranking area 44 shows the user (Amy) 441, the exerciser (Isabelle) 442 who is ranked 56th in front of the user, and the exerciser (Hettie) 443 who is ranked 58th behind the user. The exercise score of the user Amy is 73% (expressed as a percentage score). The 56th exerciser (Isabelle) scored 74%. The 58th exerciser (Hettie) scored 72%. The ranking area is based the real-time ranking of the current exercise scores, such that the ranking of the exercisers in the dynamic ranking area 44 will be changed as the exerciser's ranking is raised or fallen.

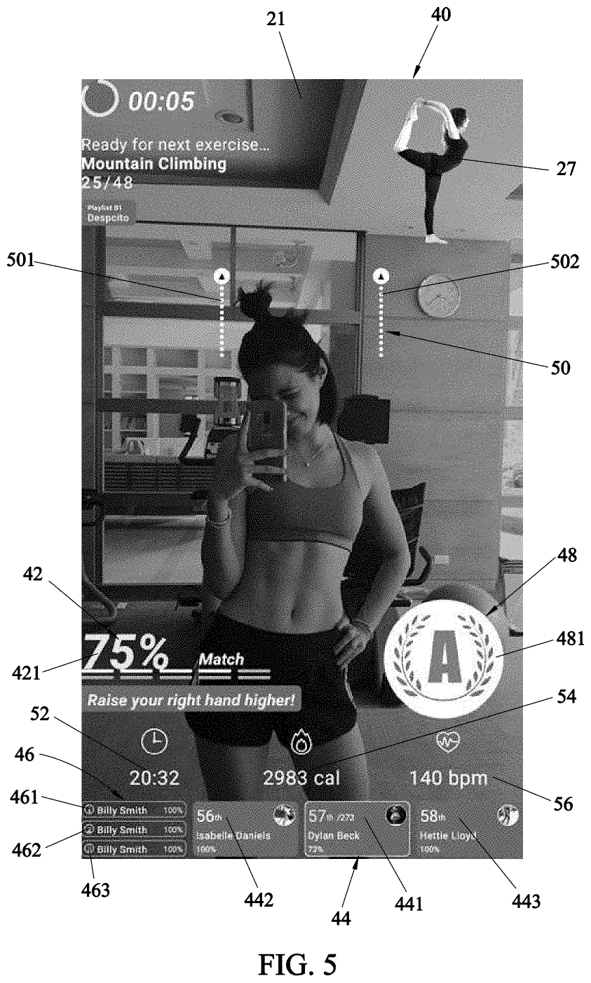

When one exerciser's exercise score is raised or fallen, their ranking will also be raised or fallen, and the ranking blocks in the dynamic ranking area 44 will be displaced horizontally, such that the exerciser with rising ranking will move forward and the exercise with falling ranking will move backward. For example, as shown in FIG. 6, when the user Amy's exercise score is increased from 73% (as shown in FIG. 4) to 75%, the current ranking of the user Amy will rise to 56th at this time, and the ranking of the former exerciser Isabelle 442 will fall from 56th to 57th, such that the order of the user Amy and the exerciser Isabelle will be changed. As shown in FIG. 7, the user Amy 441 moves forward and the exerciser Isabelle 442 moves backward, namely the rankings of the user Amy and the exerciser Isabelle are exchanged. In addition, since the dynamic ranking system of the present embodiment is configured to keep the ranking block of the user in the center of the dynamic ranking area 44, the ranking of the dynamic ranking area 44 will be changed to the order as shown in FIG. 8, namely the user Amy 441 moves to the center of the dynamic ranking area 44, the new exerciser Jane 444 who is ranked 55th in front of the user Amy 441 will move into the dynamic ranking area 44, the exerciser Isabelle 442 with current ranking of 57th will move to the location behind the user Amy 441, and the 58th exerciser Hettie 443 will be moved out of the dynamic ranking area 44. The layout of the display interface 40 is not limited by the present invention. For example, the dynamic ranking area 44 may be arranged vertically rather than horizontally. In addition, the position and size of the instructor image 27, the action scoring area 42, the ranking area 44, the leaderboard area 46 and the grade area 48 can be adjusted or changed by the user via an exercise app.

The current exercise score of an exerciser is a score for completion of an exercise class, and the exercise ranking of an exerciser is the ranking result of their exercise score compared with exercise scores of other exercisers.

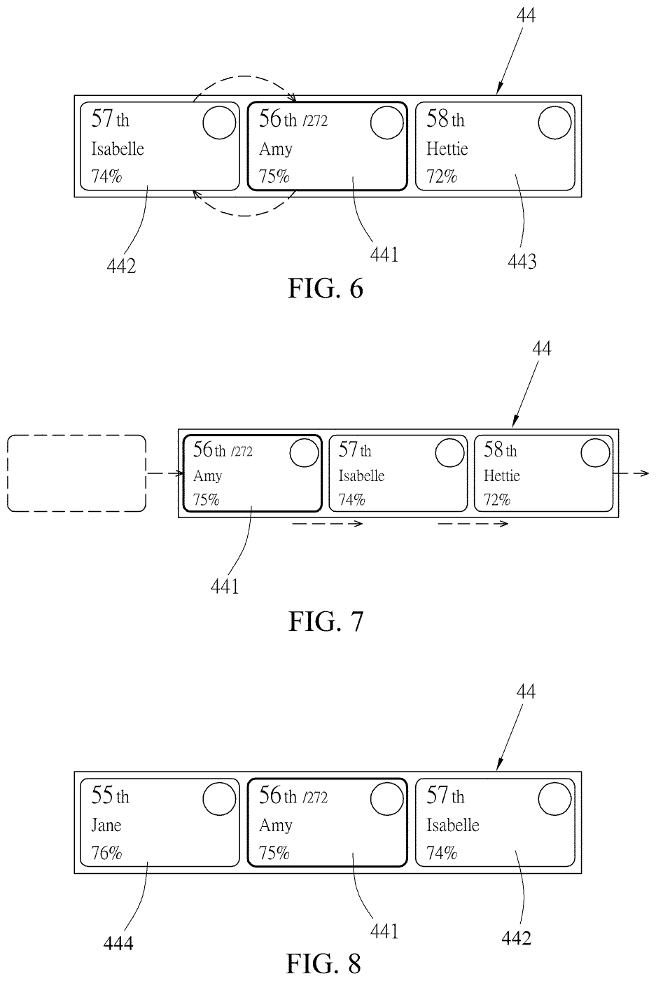

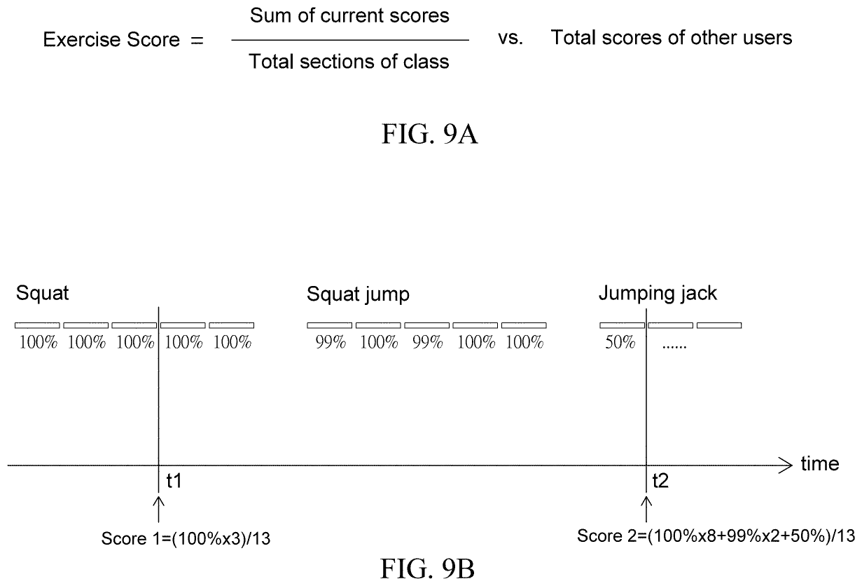

Referring to FIG. 9A, the calculation method of the (current) exercise score as represented below:

Exercise Score=Sum of current scores/Total sections of class

Ranking of exerciser=Current exercise score vs. Total scores of other users

As illustrated in FIG. 9B, assuming that the online exercise class the participants in has 13 sections, including: sections 1 to 5 are squats, sections 6 to 10 are squat jump, and sections 11 to 13 are jumping jack. The score of each section is calculated based on the aforementioned exercise score 421. For example, when the user completes the squat in the first section and their score is 100%, the user will get 100% of the exercise score in the first section. The exercise score of the user is the accumulation of the exercise score in each section.

For example, as shown in FIG. 9B, at time point t1, the user completes sections 1 to 3, and the exercise score of each section is 100%. The score 1 at the time point t1 is the cumulative score of sections 1 to 3 divided by total sections of the class, as shown in the following formula:

Score 1=(100%×3)/13=23%

At time point t2, the user completes sections 1 to 11, and the exercise scores of section 6 and section 8 are both 99%, the exercise score of section 11 is 50%, and the exercise scores of the remaining 8 sections are all 100%. The score 2 at the time point t2 is the cumulative score of sections 1 to 11 divided by total sections of the class, as shown in the following formula:

Score 2=(100%×8+99%×2+50%)/13=80.6%

Therefore, the higher the degree of completion of the exercise class, namely the more sections completed, the more exercise scores can be obtained, and thus a better ranking can be obtained. Since the exercise scores are dynamic and the exercise rankings are also dynamic, the rankings are not static. In other words, if an exerciser has a good ranking after completing an exercise class, but has not continued to participate in the exercise class, other exercisers who are more active in the exercise class will get better exercise scores and exceed their rankings. The exercise scores can be retained for a period of time (e.g. six months), which is the basis for ranking of the exerciser.

Referring to FIG. 4 and FIG. 5, the aforementioned dynamic leaderboard area 46 on the display interface 40 permanently displays at least two exercisers with the best rankings. In the preferred embodiment, the dynamic leaderboard area 46 displays the top three exercisers 461, 462, 463, so that all participants have a goal to increase their exercise scores, and the leaders have motivation to maintain their leading position. Similarly, the rankings of the top three exercisers 461, 462, and 463 will also be changed due to the rise and fall of their exercise scores.

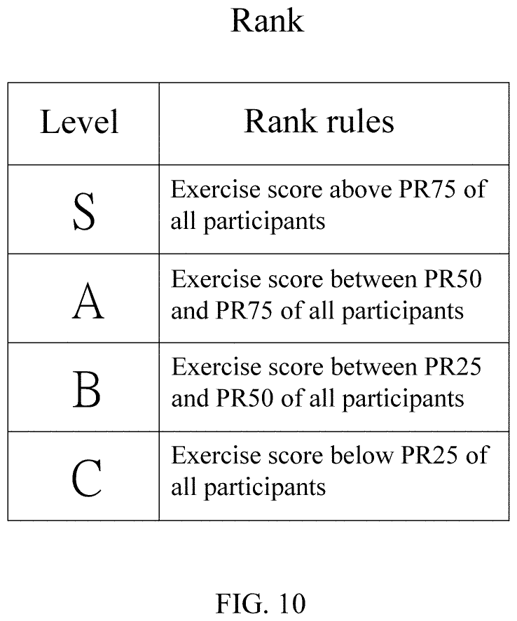

The dynamic grade area 48 ranks all current exerciser into at least three levels or grades, and displays a corresponding level 481 to let each exerciser know their current level. In the preferred embodiment, as shown in FIG. 10, all exercisers (or participants) are divided into four levels: S, A, B, and C, where level S represents that the exercise score of the exerciser is above PR75 (75th percentile) of all participants; level A represents that the exercise score of the exerciser is between PR50 (50th percentile) and PR75 (75th percentile); level B represents that the exercise score of the exerciser is between PR25 (25th percentile) and PR50 (50th percentile); level C represents that the exercise score of the exerciser is below PR25 (25th percentile).

As shown in FIG. 4, the user's dynamic level 481 is A level, namely the exercise score of the user is between PR50 and PR75. Similarly, the level (or grade) is a dynamic, real-time level, which is varied according to the user's exercise score. When the user's exercise score rises above PR75, the level will rise to S level. Conversely, when the user's exercise score falls to between PR50 and PR75, the level will fall to level B.

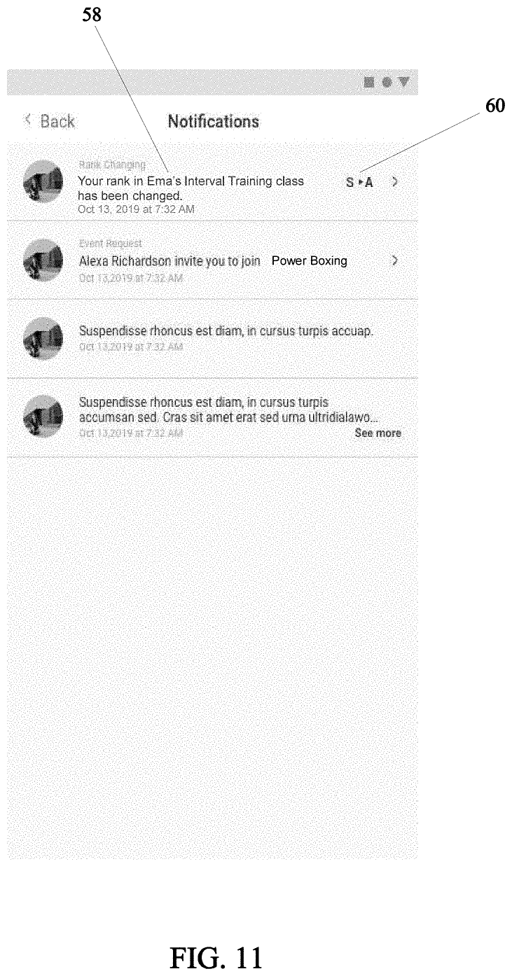

Referring to FIG. 11, when the user's dynamic level is changed, the system will send a notification to the user. The notification can be received by the user's portable device 28 (e.g. a smart phone). The notification 58 informs the user that the dynamic level has been changed, and displays the level change 60. For example, as shown in FIG. 11, the level (or rank) falls from level S to level A.

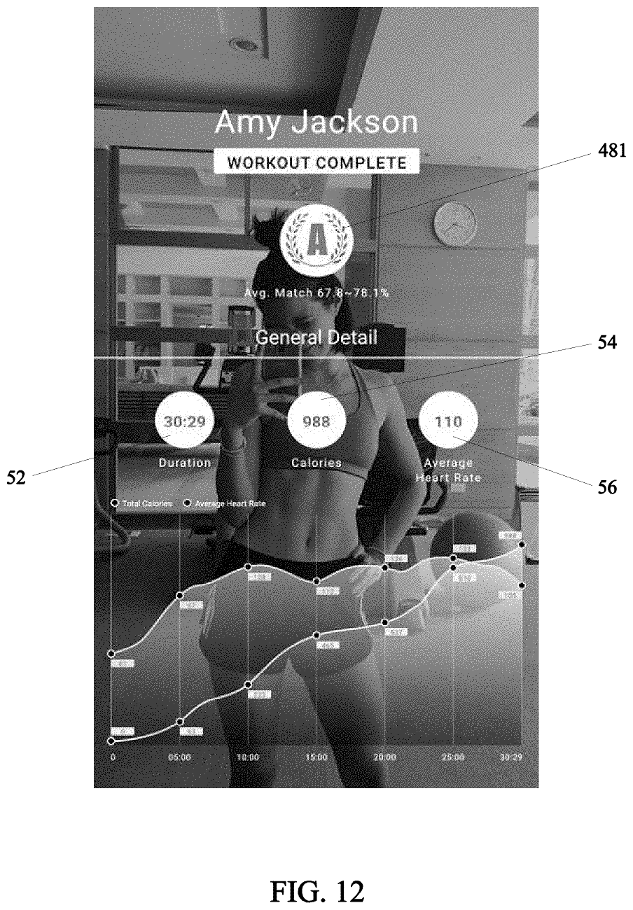

Referring to FIG. 4 and FIG. 5, the smart mirror 20 is operable to display exercise information of the user and the user's exercise ranking in all participants, including: the user's dynamic level 481, dynamic exercise ranking 441, real-time exercise score 421, and display the top three participants in the exercise class. The mirror 21 of the smart mirror 20 is also operable to display duration time 52, calorie consumption 54, and heart rate 56 of the user during the exercise class or after the exercise class. Moreover, the user can control and switch the display content of the mirror 21 through an exercise app of the portable device 28 or directly from the mirror 21. As shown in FIG. 12, in addition to the dynamic level 481, the calorie consumption and heart rate in different time periods are displayed in a graph.

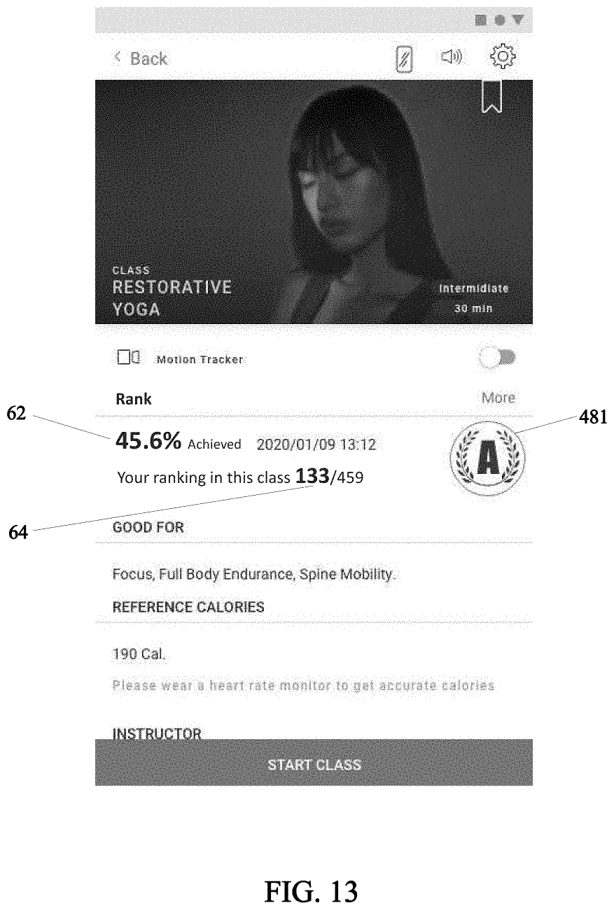

The user can review the exercise data of the exercise classes they have participated in through the exercise app on their portable device 28. As shown in FIG. 13, the user can view an online exercise class they have participated, such as exercise data of restorative yoga. The user can know the achievement rate 62 of the participated class, the ranking 64 in the class, and the level 481 in the class, and further plan their participation in this exercise class.

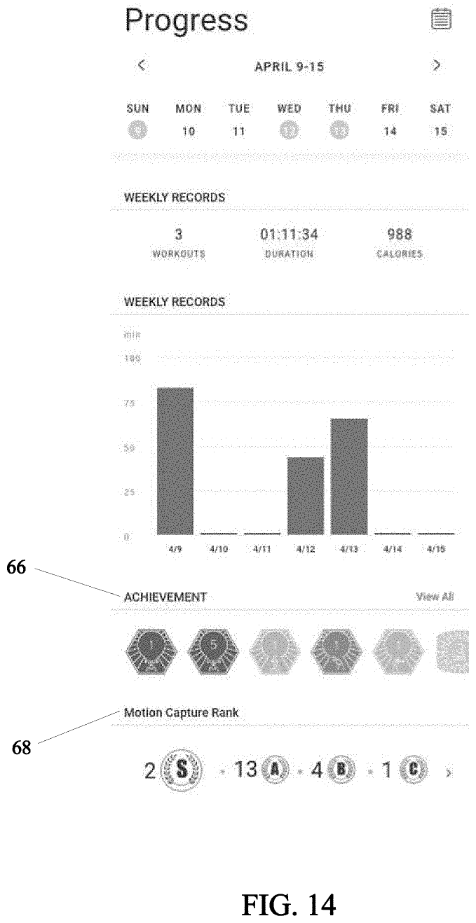

The exercise data, ranking, and exercise history of each user participated in online exercise classes are recorded and counted by the system. The exercise record can be reviewed through the exercise app. The exercise status and exercise progress within a specific time range can also be reviewed. As shown in FIG. 14, it is able to review the weekly records, including: exercise times, duration time, and calorie consumption. It can also display exercise ranking achievements 66 and the number of each level of dynamic rank 68.

The dynamic leaderboard and dynamic ranking system of the present invention is configured to provide leaderboard information and ranking information according to exercise scores of the participants. By the leaderboard information and the ranking information, the exercisers are competitive and motivated to participate in exercise classes actively for pursuit of better exercise performance and rankings.

The ranking and grade of the present invention are dynamic and real-time, and the evaluation will continue after the end of the exercise class. The ranking and grade of users will be directly proportional to their efforts to participate in online exercise classes, namely the more actively the user participates in the exercise classes, the better performance they can get.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A dynamic ranking system for showing ranking information when a user participates in an exercise class via a network, the dynamic ranking system comprising:
an exercise database, storing exercise scores of a plurality of participants who have participated in the exercise class;
a mirror display device, having a display device configured to display video content of the exercise class, the video content including a guiding image for guiding the user to perform a workout when the user participates in the exercise class;
a motion capturing unit configured to capture movement of the user through a camera device; and
a comparing unit configured to determine a matching degree between the movement of the user and movement of the guiding image to obtain an exercise score of the user, and to identify a grade of the user according to the exercise score.

2. The dynamic ranking system as claimed in claim 1, wherein the comparing unit is configured to rank the exercise scores of all participants in the exercise class into at least three levels, and display the level to which the current exercise score of the user belongs on the mirror display device.

3. The dynamic ranking system as claimed in claim 1, wherein the comparing unit is configured to rank the participants in the exercise class according to their exercise scores into at least three levels including a high level, a middle level, and a low level; and wherein the level of the user is displayed on the mirror display device.

4. The dynamic ranking system as claimed in claim 1, wherein the exercise scores of all participants are divided into four levels including: a first level where the exercise score is above 75th percentile of all participants, a second level where the exercise score is between 50th percentile and 75th percentile of all participants, a third level where the exercise score is between 25th percentile and 50th percentile of all participants, and a fourth level where the exercise score is below 25th percentile of all participants.

5. The dynamic ranking system as claimed in claim 1, wherein the mirror display device further has a mirror disposed in front of the display device and the video content being visible through the mirror, the mirror configured to reflect an image of the user opposite the mirror, so that the image of the user reflected by the mirror and the guiding image are shown on the mirror at the same time during the exercise class.

6. The dynamic ranking system as claimed in claim 1, wherein the grade of the user is dynamically changed when ranking of the user in the exercise class is changed.

7. The dynamic ranking system as claimed in claim 1, wherein the guiding image comprising an instructor image demonstrating exercise movements of the exercise class to allow the user to follow, and the comparing unit is configured to determine the matching degree between the movement of the user and the movement of the instructor image to obtain the exercise score of the user.

8. The dynamic ranking system as claimed in claim 1, wherein the guiding image comprising a guide track for guiding the user to perform exercise movements of the exercise class, and the comparing unit is configured to determine the matching degree between the movement of the user and the guide track to obtain the exercise score of the user.

9. The dynamic ranking system as claimed in claim 1, wherein the motion capturing unit comprises a camera device configured capture an image of the user in front of the mirror display device for tracking movement of the user.

10. The dynamic ranking system as claimed in claim 1, wherein the exercise class has a plurality of sections, and the exercise score of the user is based on accumulation of the exercise score of each section in the exercise class the user has finished.

11. The dynamic ranking system as claimed in claim 1, wherein the mirror display device is configured to display the matching degree of the user in a dynamic action scoring area, a ranking of the user in a dynamic ranking area, and the grade of the user in a dynamic grade area; and wherein the ranking of the user is a ranking of their exercise score compared with exercise scores of other participants in the exercise class.
